(19) United States
(12) Patent Application Publication
(10) Pub. No.: US 2025/0335913 A1
(43) Pub. Date: Oct. 30, 2025

(54) TECHNOLOGIES FOR ESTABLISHING PROOF OF INVENTORY TRANSACTION

(71) Applicant: VETSNAP CORP., Thousand Oaks, CA (US)

(72) Inventors: Steven Patrick Elliott, Thousand Oaks, CA (US); Yangyang Shi, Thousand Oaks, CA (US)

(21) Appl. No.: 19/193,713

(22) Filed: Apr. 29, 2025

Related U.S. Application Data
(60) Provisional application No. 63/639,953, filed on Apr. 29, 2024.

(51) Int. Cl. G06Q 20/40; G06Q 10/087; H04L 9/32

(52) U.S. Cl. CPC G06Q 20/401; G06Q 10/087; H04L 9/3236

(57) ABSTRACT

Technologies for establishing an attestable proof of witness for a controlled substance transaction entry include a first computing device that establishes device communications with a second computing device. The first computing device executes an application associated with a software platform server. The device communications are established in response to a generation of a controlled substance transaction entry. A proof of witness is generated as a function of the established device communications and a controlled substance associated with the controlled substance transaction entry. The proof of witness is associated with the controlled substance transaction entry.

### CROSS-REFERENCE TO RELATED APPLICATIONS

The present application claims priority to U.S. Prov. Pat. Appl. No. 63/639,953, filed Apr. 29, 2024, which is incorporated by reference in its entirety.

### FIELD

Embodiments disclosed herein generally relate to practice management and inventory systems, and more specifically, to establishing a regulation compliant proof of witness for an inventory transaction.

### BACKGROUND

In the medical and veterinary industry, a practice (e.g., a clinic, hospital, pharmacy, etc.) must, by law, record and track usage of controlled substances in patients. Generally, controlled substances are chemicals, pharmaceutical agents, and the like that have been identified by a governmental body as having a potential for abuse and dependency. For example, the United States Drug Enforcement Agency categorizes and regulates controlled substances based on medicinal use, potential for abuse, and safety or dependence liability. The practice generally maintains the controlled substance records in a controlled substance log, which is subject to various requirements by regulatory agencies. Current technologies allow a medical or veterinary practice to electronically maintain controlled substance inventories. For example, cloud-based solutions exist that enable practitioners at a given veterinary clinic to manage the controlled substance log remotely, such as from different locations within the veterinary practice via different computing devices.

### SUMMARY

One embodiment presented herein discloses a method for establishing a proof of witness for a controlled substance transaction. The method generally includes establishing, by a first computing device, device communications with a second computing device. The first computing device executes an application associated with a software platform server. The device communications are established in response to a generation of a controlled substance transaction entry. The method also generally includes generating, as a function of the established device communications and a controlled substance associated with the controlled substance transaction entry, a proof of witness between the first computing device and the second computing device of the controlled substance transaction entry. The proof of witness is associated with the controlled substance transaction entry. The controlled substance transaction entry and proof of witness are transmitted to the software platform server for verification and storage.

Another embodiment presented herein discloses a computer-readable storage medium storing instructions, which, when executed on one or more processors, causes a first computing device executing an application associated with a software platform server to establish device communications with a second computing device. The device communications are established in response to a generation of a controlled substance transaction entry. The first computing device generates as a function of the established device communications and a controlled substance associated with the controlled substance transaction entry, a proof of witness between the first computing device and the second computing device of the controlled substance transaction entry. The proof of witness is associated with the controlled substance transaction entry. The controlled substance transaction entry and proof of witness are transmitted to the software platform server for verification and storage.

Yet another embodiment presented herein discloses a computing device. The computing device has one or more processors and a memory storing instructions which causes the computing device to establish device communications with a second computing device. The device communications are established in response to a generation of a controlled substance transaction entry. The first computing device generates as a function of the established device communications and a controlled substance associated with the controlled substance transaction entry, a proof of witness between the first computing device and the second computing device of the controlled substance transaction entry. The proof of witness is associated with the controlled substance transaction entry. The controlled substance transaction entry and proof of witness are transmitted to a software platform server for verification and storage.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 illustrates a simplified conceptual diagram of a computing environment in which a remote witness proof for an inventory transaction is established, according to an embodiment;

FIG. 2 is a simplified block diagram of the platform server described relative to FIG. 1, according to an embodiment;

FIG. 3 is a simplified block diagram of a mobile device configured to establish a remote witness proof for an inventory transaction, according to an embodiment;

FIG. 4 is a conceptual block diagram illustrating components of the inventory management application described relative to FIG. 1, according to an embodiment;

FIG. 5 is a conceptual diagram illustrating a system for generating a regulation-compliant remote witness proof to associate with an inventory transaction, according to an embodiment; and

FIG. 6 is a sequence diagram for generating a regulation-compliant remote witness proof to associate with an inventory transaction.

### DETAILED DESCRIPTION

Some organizational inventory policies and governmental regulations require that a witness is present when certain inventory is taken out or otherwise spent. For example, controlled substance regulations require that a witness to a controlled substance transaction (e.g., a transaction in which a controlled substance is administered or dispensed and requires signature by a witness individual to record the transaction) provide proof that he or she observed an individual administering or otherwise dispensing a controlled substance for a patient.

In the United States, proof of witness for a controlled substance transaction may be obtained remotely to comply with regulations, such as through a video call to the witness conducted on one's phone. However, concerns exist with prior approaches for generating, recording, and certifying remote witness data. For example, prior approaches merely require the entry of a user (e.g., a veterinary practitioner) indicating that the witnessing via a video call took place, in which the entry can also include the uploading of video proof. However, such prior approaches are unable to attest that the video call actually took place at the time that the user purported on the log. Further, other situations exist in which extra proof of witness may be desired to confirm that the witness had been a part of the controlled substance transaction.

Prior technologies in which digital controlled substance logbooks are maintained include features that will allow a witness on-site to furnish a signature or other witness proof that a controlled substance was dispensed in a given transaction. However, an issue that might arise is that sometimes a medical practitioner does not physically have a witness present or a witness capable of signing the transaction in the digital logbook (e.g., because the witness does not have access to the digital logbook). For instance, in some cases, a veterinary practice may dispatch a veterinary practitioner to a remote location to administer or dispense a controlled substance to an animal patient, such as to a horse. Occasionally, the practitioner is the only individual on-site, which can happen if the owners are away from the site. In such an event, obtaining witness proof is difficult unless the medical practitioner physically brings a witness to the site while administering or dispensing the controlled substance to the animal patient.

Additionally, a stable and high-speed connection is often necessary for clear and uninterrupted video and audio to ensure that a given witness is able to observe the administration of controlled substances. Previous approaches often rely on low-resolution video technologies or difficulty in maintaining network connections, which cause issues in readability of medication labels and dosages when rendered on a device display, as well as the witness missing critical steps due to latency issues (e.g., lag, video freeze). Further, previous approaches often have been insufficient in ensuring compliant data storage (e.g., by storing video and logs in unsecured data storage systems such as non-HIPAA-compliant cloud services), risking data leakage to unauthorized parties.

To address such issues, embodiments presented herein disclose technological improvements for establishing proof of witness for an inventory transaction, such as a controlled substance transaction maintained in a digital controlled substance log. As further described herein, the present disclosure addresses such concerns by generating an attestable proof of witness based on various electronic sources that corroborate the witnessing of the controlled substance transaction.

The disclosed technological improvements may be incorporated in an inventory management system platform that integrates with a practice inventory management system (PIMS) of a medical or veterinary practice. The platform may serve as a source of inventory data, such as controlled substance usage data, and provide a digital controlled substance usage log (also referred to herein as a “digital logbook”) that the veterinary practice can use, e.g., via a client device executing a platform application thereon, to enter usage data throughout the day while treating patients and remain compliant with legal and regulatory requirements.

The platform application enables a user (e.g., a pharmacist, medical practitioner, veterinary practitioner, etc.) to record, in the digital logbook, transactions in which the user administered or otherwise dispensed of an inventory item, such as of a controlled substance to a patient. To remain compliant with legal and regulatory requirements, the platform application also provides an interface in which a user may contemporaneously record, upload, or otherwise provide digital media proof that a witness observed the administration or dispensing of the controlled substance, such as a scan of the signature or electronic signature of the witness. To establish attestable proof of the remote witness, metadata associated with the digital media can be associated with an underlying record, such as a timestamp, digital signature information associated with each device in the transaction, device and device component data, geolocation data, access logs, and the like.

Advantageously, the disclosed technology improves upon prior technological approaches for obtaining witness proofs for the administration of controlled substances to a patient. The technologies disclosed herein provide an attestable remote digital witness proof that can be securely stored and associated with a controlled substance transaction in a digital logbook. The disclosed technologies may secure proofs based on metadata generated based on the transaction and by employing encryption methods throughout the capture of the proof. In addition, the recorded and transmitted data that is incorporated into a proof are encrypted using strong algorithms and transmitted over secure transmissions to maintain compliance with health data privacy laws and controlled substance regulations.

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

Note, the following uses controlled substance transactions as a reference example for generating a witness proof that an inventory transaction has taken place. However, one of skill in the art will recognize that the broader inventive concept of generating a unique witness proof from various forms of metadata and device information can be adapted to a variety of inventory-based transactions, such as the dispensing or administering of substances at a medical practice.

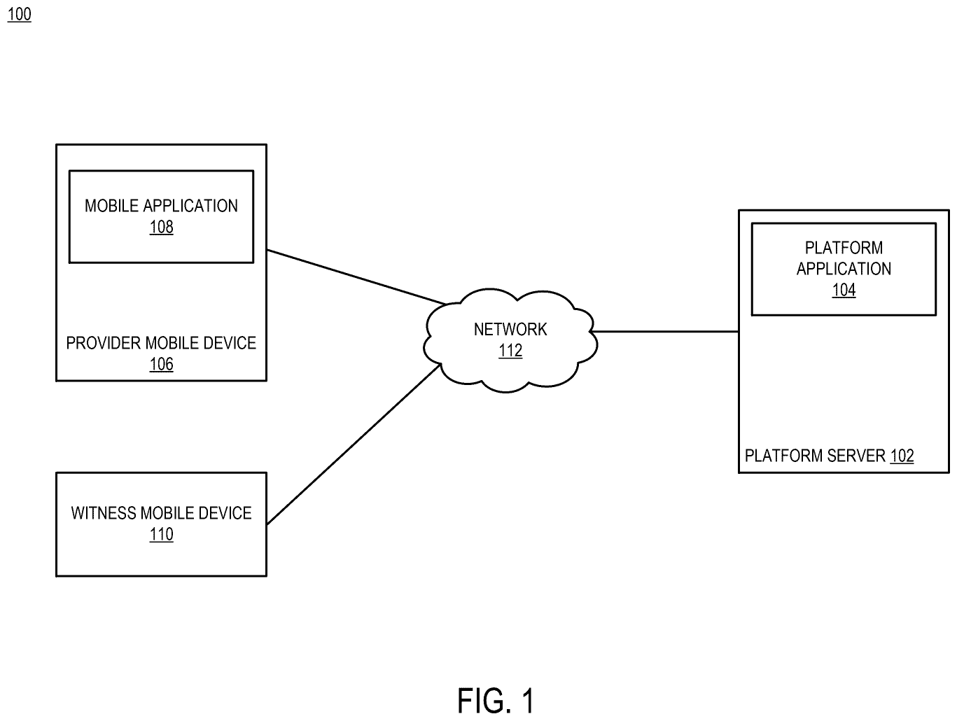

Referring now to FIG. 1, a computing environment 100 in which an attestable proof of witness for a controlled substance transaction may be established, according to an embodiment. As shown, the computing environment 100 includes a platform server 102, provider mobile device 106, and a witness mobile device 110, each interconnected via a network 112 (e.g., the Internet, a local area network, private network, etc.).

The platform server 102 may correspond to a controlled substance inventory management platform system that is accessible by the provider mobile device 106 and the witness mobile device 110. The platform server 102 includes a platform application 104 configured to perform the functions disclosed herein. For example, the platform server 104 may generate and manage operations for a digital controlled substance logbook which tracks the dispensing, administration, and usage of controlled substances within a given medical practice (e.g., a veterinary clinic, pharmacy, hospital, healthcare facility, research institution, etc.) so as to comply with recordkeeping requirements set by regulatory agencies. The digital logbook may contain fields that stores transaction information, such as a timestamp of when the transaction occurred, patient information, prescription information, medical professional information, amount dispensed information, a remaining balance of the controlled substance, and signatures such as from the medical professional administering or otherwise dispensing the controlled substance.

The platform server 102 may be embodied as a physical computing system or a virtual computing instance executing on a cloud provider network. In an embodiment, the platform server 102 may be centrally located (e.g., on-premise within the medical practice) or can comprise multiple servers 112 hosted in separate geographical locations. In an embodiment, different functions of the platform server 102 may be carried about by individual or groups of computing systems.

The illustrative provider mobile device 106 represents a mobile device of a user, such as a medical or veterinary practitioner, conducting a controlled substance transaction. The provider mobile device 106 includes a mobile application (“app”) that provides a user interface enabling access to the platform application 104, e.g., to record a controlled substance transaction in a digital logbook. The illustrative witness mobile device 110 represents a mobile device of an individual that may or may not have access to the platform application 104 but is used in establishing a proof of witness of a controlled substance transaction.

In an embodiment, the provider mobile device 106 and the witness mobile device 106 may be embodied as any physical computing device such as a smartphone, tablet device, wearable device, and the like, that is configured to perform the functions described herein. In other embodiments, the provider mobile device 106 and witness mobile device 110 need not be a portable or mobile device, per se, but any physical computing device (or virtual computing instance) capable of performing the aforementioned functions, such as conducting real-time video communications with other devices or providing an indication (such as a signature) that an individual administered, dispensed, or witnessed the administering or dispensing of a controlled substance.

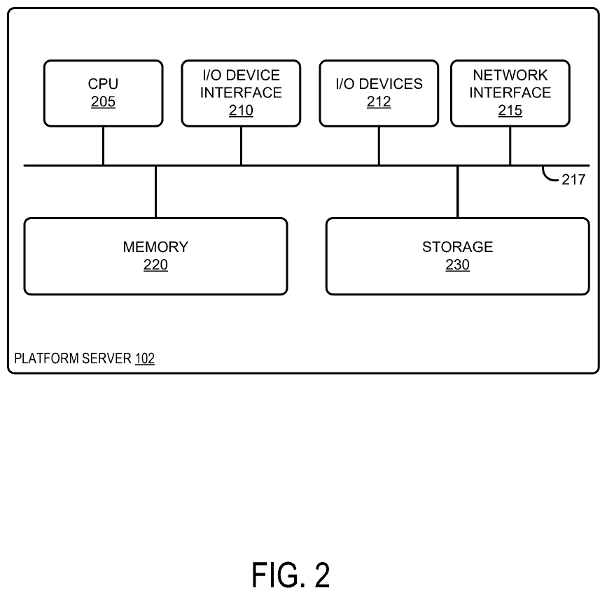

Referring now to FIG. 2, a block diagram illustrating components of the platform server 102. As shown, platform server 102 includes, without limitation, a central processing unit (CPU) 205, an input/output (I/O) device interface 210, one or more I/O devices 212, a network interface 215, a memory 220, and a storage 230, each interconnected via a hardware bus 217. Of course, the actual platform server 102 will include a variety of additional hardware components not shown. Additionally, in some embodiments, one or more of the illustrative components may be incorporated in, or otherwise form a portion of, another component.

The CPU 205 retrieves and executes programming instructions stored in the memory 220 (e.g., of the platform application 112). The CPU 205 may be embodied as one or more processors, each processor being a type capable of performing the functions described herein. For example, the CPU 205 may be embodied as a single or multi-core processor(s), a microcontroller, or other processor or processing/controlling circuit. In some embodiments, the CPU 205 may be embodied as, include, or be coupled to a field programmable gate array (FPGA), an application-specific integrated circuit (ASIC), reconfigurable hardware or hardware circuitry, or other specialized hardware to facilitate performance of the functions described herein. The hardware bus 217 is used to transmit instructions and data between the CPU 205, storage 230, network interface 215, and the memory 220. CPU 205 is included to be representative of a single CPU, multiple CPUs, a single CPU having multiple processing cores, and the like. The memory 220 may be embodied as any type of volatile (e.g., dynamic random access memory, etc.) or non-volatile memory (e.g., byte addressable memory) or data storage capable of performing the functions described herein. Volatile memory may be a storage medium that requires power to maintain the state of data stored by the medium. Non-limiting examples of volatile memory may include various types of random access memory (RAM), such as DRAM or static random access memory (SRAM). One particular type of DRAM that may be used in a memory module is synchronous dynamic random access memory (SDRAM). In particular embodiments, DRAM of a memory component may comply with a standard promulgated by JEDEC, such as JESD79F for DDR SDRAM, JESD79-2F for DDR2 SDRAM, JESD79-3F for DDR3 SDRAM, JESD79-4A for DDR4 SDRAM, JESD209 for Low Power DDR (LPDDR), JESD209-2 for LPDDR2, JESD209-3 for LPDDR3, and JESD209-4 for LPDDR4. Such standards (and similar standards) may be referred to as DDR-based standards and communication interfaces of the storage devices that implement such standards may be referred to as DDR-based interfaces.

The network interface 215 may be embodied as any hardware, software, or circuitry (e.g., a network interface card) used to connect the platform server 102 over the network 112 and providing the network communication component functions described above. For example, the network interface 215 may be embodied as any communication circuit, device, or collection thereof, capable of enabling communications over the network 112 between the platform server 102 and other devices (e.g., the provider mobile device 106, witness mobile device 110, etc.). The network interface 215 may be configured to use any one or more communication technology (e.g., wired, wireless, and/or cellular communications) and associated protocols (e.g., Ethernet, Bluetooth®, Wi-Fi®, WiMAX, 5G-based protocols, etc.) to effect such communication. For example, to do so, the network interface 215 may include a network interface controller (NIC, not shown), embodied as one or more add-in-boards, daughtercards, controller chips, chipsets, or other devices that may be used by the platform server 102 for network communications with remote devices. For example, the NIC may be embodied as an expansion card coupled to the I/O device interface 210 over an expansion bus such as PCI Express.

The I/O device interface 210 allows the I/O devices 212 to communicate with hardware and software components of the platform server 102. For example, the I/O device interface 210 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O device interface 210 may form a portion of a system-on-a-chip (SoC) and be incorporated, along with one or more of the CPU 205, the memory 220, and other components of the platform server 102.

The I/O devices 212 may be embodied as any type of I/O device connected with or provided as a component to the platform server 102. I/O devices such as keyboards, mice, and printers may be included as I/O devices 212 (e.g., to access an administrator UI, print controlled substance transaction records, etc.).

The storage 230 may be embodied as any type of devices configured for short-term or long-term storage of data such as, for example, memory devices and circuits, memory cards, hard disk drives (HDDs), solid-state drives (SSDs), or other data storage devices. The storage 230 may include a system partition that stores data and firmware code for the storage 230. The storage 230 may also include an operating system partition that stores data files and executables for an operating system.

In addition, the storage 230 may embodied as a secure storage capable of storing metadata associated with witness proof information, such as hashes, timestamps, geolocation data, and device information. Data stored in the storage 230 may be encrypted and/or isolated to ensure compliance with health data privacy laws.

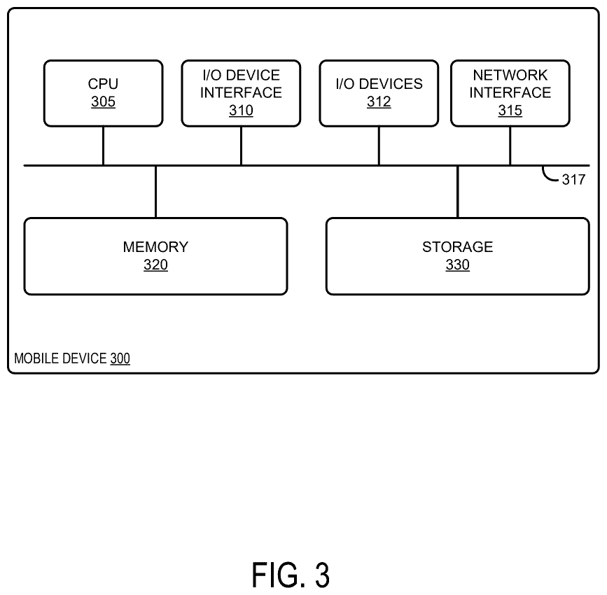

Referring now to FIG. 3, a block diagram depicting various components of a mobile device 300 (representative of the provider mobile device 106 or witness mobile device 110) is shown. As shown, mobile device 300 includes, without limitation, a central processing unit (CPU) 305, an input/output (I/O) device interface 310, one or more I/O devices 312, a network interface 315, a memory 320, and a storage 330, each interconnected via a hardware bus 317.

Of course, the actual mobile device 300 will include a variety of additional hardware components not shown. Additionally, in some embodiments, one or more of the illustrative components may be incorporated in, or otherwise form a portion of, another component. Further, each of the CPU 305, I/O interface 310, I/O devices 312, network interface 315, memory 320, and storage 330 are similar to the respective components described relative to FIG. 2.

In an embodiment, the I/O devices 312 preferably include a one or more audiovisual capture devices (e.g., a front-facing camera, rear camera, audio capture device, etc.). The audiovisual capture devices may also include depth sensors or multi-lens arrays to provide richer metadata such as depth map (per frame or per keyframe), focal length, lens distortion, object distance, and the like. In addition, the mobile device 300 may include a secure clock therein that is synchronized with some trusted timestamp source, such as GPS satellites.

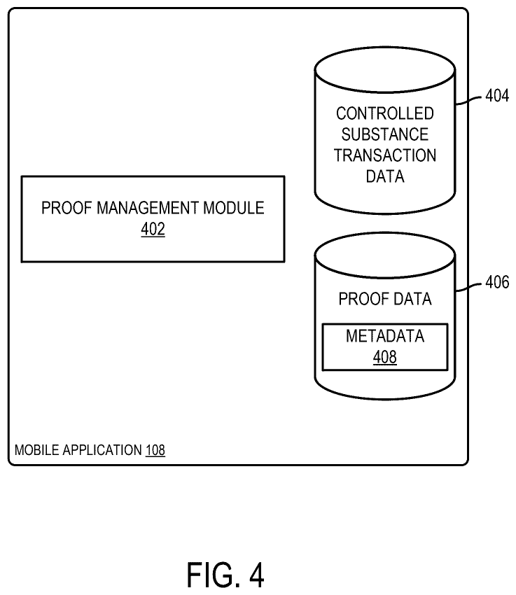

Referring now to FIG. 4, a conceptual diagram of software components of the mobile application 108 are shown. As shown, the mobile application 108 may include a proof management module 402, one or more controlled substance transactions 404, and proof data 406. The proof management module 402 is configured to perform one or more functions for establishing an attestable proof of witness of an individual dispensing or administering a controlled substance (or otherwise conducted a controlled transaction) to a patient at a given location and at a given time, and generating and storing proof data 406 representative of the same. The proof management module 402 may link a given witness proof with a respective controlled substance transaction (e.g. by appending the relevant proof data 406 with a respective entry associated with the controlled substance transaction 404).

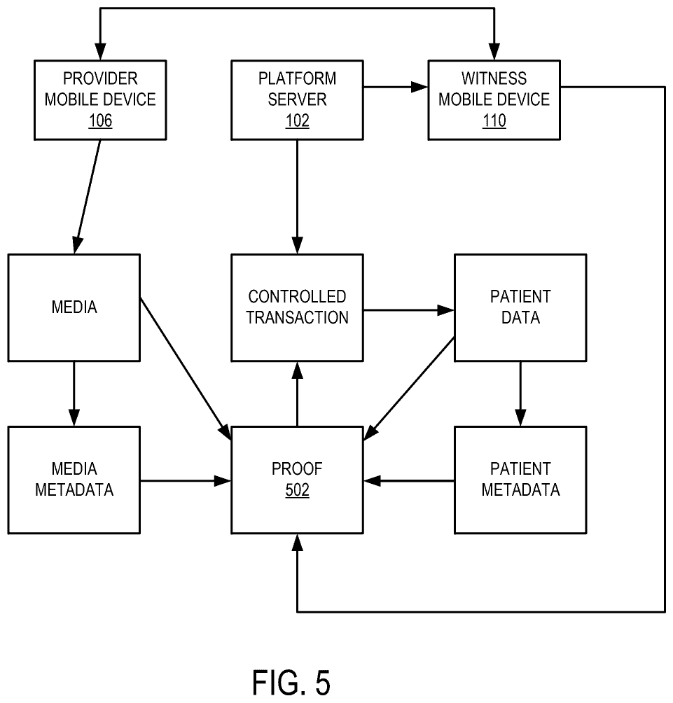

Referring now to FIG. 5, a conceptual diagram illustrating a system for generating a witness proof to associate with a controlled substance is shown. As shown, a proof 502 may be generated as a function of various sources. For example, the proof 502 may be generated as a function of media output by the provider device 106 (e.g., audio/video content recorded by the provider device 106 during a given controlled substance transaction) and metadata associated with that media. Metadata associated with media can include timestamps (e.g., creation date, modification date), device information (e.g., information regarding a camera used in the mobile device to capture a proof, make and model of the mobile device, etc.), geolocation, software history, hashes and digital signatures, length of the media, and the like.

Further, the proof 502 may be generated as a function of patient data and metadata. The patient data may be obtained from the platform server 102 (e.g., an electronic record associated with the patient's chart) and also from the recorded media content. Patient data that can be used to generate the proof in part include medical record information (e.g., name of the patient, name of the owner, address information, other data provided in the record, etc), patient identification (e.g., identifying indicators associated with the patient, such as a description of the patient, wristband information, etc.). Metadata associated with the patient data can include timestamp information associated to when the medication was administered, format information, system properties, etc.), corroborative data sent by the witness device 110 (e.g., device information, geolocation information, timestamp information, etc.), and so on. Illustratively, the proof may be linked to or otherwise associated with a given controlled substance transaction entry for an underlying digital logbook of a medical practice.

Patient data and metadata can include medical record information (e.g., which can include time and date information that the medication was administered), patient identification (e.g., identifying indicators associated with the patient, such as a description of the patient, wristband information, etc.).

The data from the media, media metadata, patient data, patient metadata, and the like may be used as input for a secure hash function (e.g., SHA-256, BLAKE3, double hashing, etc.) to generate the proof 502. The use of a hash function ensures data integrity of the proof and that the inputs cannot be reverse engineered or tampered once generated. Once generated, the proof may be uploaded and/or otherwise associated with the underlying controlled transaction in the digital logbook.

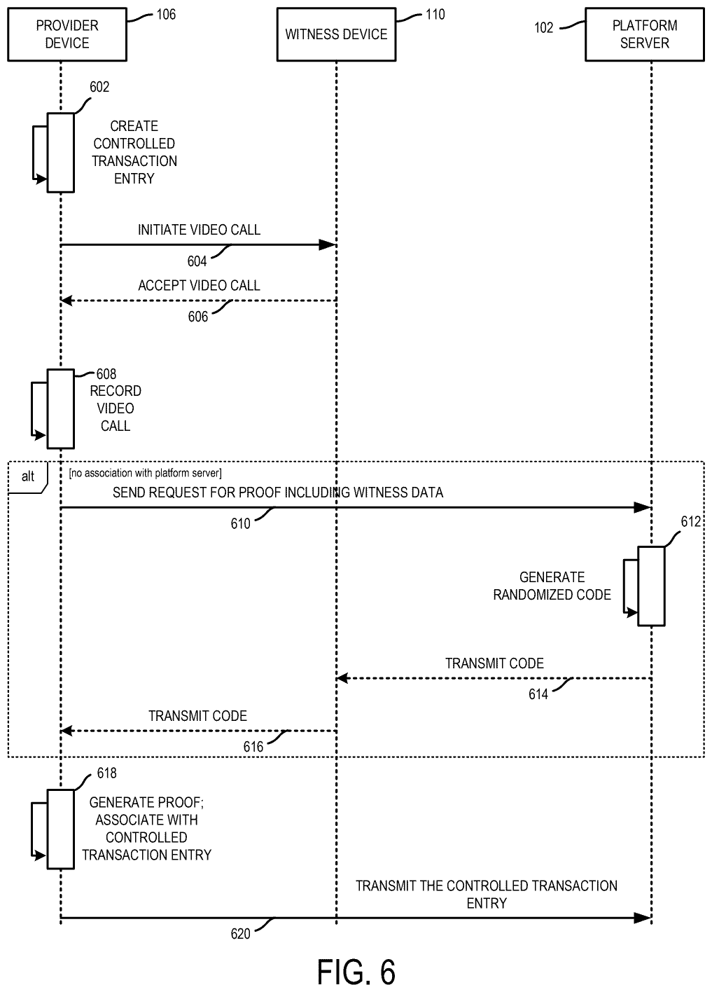

Referring now to FIG. 6, a sequence for generating a witness proof for a given controlled substance transaction is shown. Illustratively, the sequence depicts actions taking place between the provider device 106, witness device 110, and platform server 102.

At 602, the provider device 106 creates a controlled transaction entry in the digital logbook of the mobile application 108. In this example, the provider device 106 may do so in response to a user (e.g., a medical or veterinary provider) dispensing or administering a controlled substance to a patient.

At 604, the provider device 106 initiates a video call with the witness device 110. At 606, the witness device 110 accepts the video call. Once the video call is established, at 608, the provider device 106 begins recording the call (e.g., by accessing the camera and microphone functions of the provider device 106 via the mobile application 108).

Alternatively or in addition to the video call (e.g., during the video call), in 610, the provider device 106 sends a request to the platform server 102 for proof of witness of the controlled substance transaction. For example, the provider device 106 may do so in the event that the witness device 110 does not have a mobile application 108 installed (and otherwise has no association with the platform server 102). In turn, at 612, the platform server 102 generates a randomized code such as a one-time password, random nonce, random session key, and so on.

At 614, the platform server 102 transmits the randomized code (e.g., via a SMS text message, email message, authenticator application message, and the like). Once received, at 616, the witness device 110 transmits the randomized code to the provider device 106. For example, the witness device 110 may forward the code by text message to a phone number associated with the provider device 106. As another example, the witness device 110 may access a website associated with the platform server 102 (or provider device 106) and input the randomized code in a prompt provided by the website. As yet another example, the witness may provide the number to the provider, and in turn the provider may input the randomized code into the provider device 106 manually through a prompt.

In an embodiment, the randomized code that is received from the platform server 102 by the witness device 110 and thereafter sent to the provider device 106 may be used used as a basis to establish that the provider and witness (along with their devices 106 and 110) had established device communications with one another at a given point in time when the controlled substance transaction occurred. The randomized code may be a universally unique code that may be linked to a specific controlled substance transaction.

Once the code has been received by the provider device 106, at 618, the provider device 106 may generate a proof of witness for the specific controlled substance transaction and associate the proof with the controlled substance transaction entry in the digital logbook. For example, as stated, the provider device 106 may generate the proof as a function of various aspects of the controlled substance transaction event, such as mobile device data and metadata associated with, recorded video call data and metadata, patient data and metadata, practitioner information, the generated randomized code, geolocation data, universal timestamp data, device address information, controlled substance information (e.g., container identifier information, substance identifier information), employer ID information, and the like may be used to generate a proof of witness. For instance, such aspects may be used as inputs for a hash function to generate the proof. At 620, the provider device 106 transmits the controlled transaction entry to the platform server 102. In turn, the platform server 102 may verify the veracity of the entry, and once verified, stores the transaction entry in a data store.

Note, the above references video calls as a means of establishing that the witness and provider were conducting device communications at the same time that a controlled substance transaction was being conducted by the provider. Of course, one of skill in the art will recognize that the embodiments of the present disclosure may adapt other features associated with communications between two devices in proximity to one another to establish a proof of witness for the purpose of a controlled substance transaction. For example, wireless communication pairing mechanisms may be used in combination with universal timestamping may be used to establish that the witness and provider devices were in proximity to one another at the time of the controlled substance transaction.

While the foregoing is directed to embodiments of the present disclosure, other and further embodiments of the disclosure may be devised without departing from the basic scope thereof, and the scope thereof may be determined by the example claims that follow.

1. A computer-implemented method for establishing a proof of witness for a controlled substance transaction, the method comprising:
establishing, by a first computing device, device communications with a second computing device, wherein the first computing device executes an application associated with a software platform server, wherein the device communications are established in response to a generation of a controlled substance transaction entry;
generating, as a function of the established device communications and a controlled substance associated with the controlled substance transaction entry, a proof of witness between the first computing device and the second computing device of the controlled substance transaction entry;
associating the proof of witness with the controlled substance transaction entry; and
transmitting the controlled substance transaction entry and the proof of witness to the software platform server for verification and storage.

2. The computer-implemented method of claim 1, wherein the controlled substance transaction entry is entered into a digital logbook managed by the software platform server.

3. The computer-implemented method of claim 1, wherein the first computing device is in a different geographical location from the second computing device.

4. The computer-implemented method of claim 1, wherein the proof of witness is generated further as a function of metadata associated with the first computing device and the second computing device.

5. The computer-implemented method of claim 1, wherein the established device communications comprises a media communication, and wherein the proof of witness is generated further as a function of metadata associated with the media communication.

6. The computer-implemented method of claim 5, wherein the established metadata associated with the media communication comprises geolocation data, timestamp data, and recording device data.

7. The computer-implemented method of claim 1, wherein the proof of witness is generated using a hash function.

8. A computer-readable storage medium storing a plurality of instructions, which, when executed by one or more processors, causes a first computing device executing an application associated with a software platform server to:
establish device communications with a second computing device, wherein the device communications are established in response to a generation of a controlled substance transaction entry;
generate, as a function of the established device communications and a controlled substance associated with the controlled substance transaction entry, a proof of witness between the first computing device and the second computing device of the controlled substance transaction entry;
associate the proof of witness with the controlled substance transaction entry; and
transmit the controlled substance transaction entry and the proof of witness to the software platform server for verification and storage.

9. The computer-readable storage medium of claim 8, wherein the controlled substance transaction entry is entered into a digital logbook managed by the software platform server.

10. The computer-readable storage medium of claim 8, wherein the first computing device is in a different geographical location from the second computing device.

11. The computer-readable storage medium of claim 8, wherein the proof of witness is generated further as a function of metadata associated with the first computing device and the second computing device.

12. The computer-readable storage medium of claim 8, wherein the established device communications comprises a media communication, and wherein the proof of witness is generated further as a function of metadata associated with the media communication.

13. The computer-readable storage medium of claim 12, wherein the established metadata associated with the media communication comprises geolocation data, timestamp data, and recording device data.

14. The computer-readable storage medium of claim 8, wherein the proof of witness is generated using a hash function.

15. A computing device, comprising:
one or more processors; and
a storage comprising a plurality of instructions, which, when executed on the one or more processors, causes the computing device to:
establish device communications with a second computing device, wherein the device communications are established in response to a generation of a controlled substance transaction entry;
generate, as a function of the established device communications and a controlled substance associated with the controlled substance transaction entry, a proof of witness between the computing device and the second computing device of the controlled substance transaction entry;
associate the proof of witness with the controlled substance transaction entry; and
transmit the controlled substance transaction entry and the proof of witness to a software platform server for verification and storage.

16. The computer-readable storage medium of claim 8, wherein the controlled substance transaction entry is entered into a digital logbook managed by the software platform server.

17. The computer-readable storage medium of claim 8, wherein the computing device is in a different geographical location from the second computing device.

18. The computer-readable storage medium of claim 8, wherein the proof of witness is generated further as a function of metadata associated with the computing device and the second computing device.

19. The computer-readable storage medium of claim 8, wherein the established device communications comprises a media communication, and wherein the proof of witness is generated further as a function of metadata associated with the media communication, and wherein the established metadata associated with the media communication comprises geolocation data, timestamp data, and recording device data.

20. The computer-readable storage medium of claim 8, wherein the proof of witness is generated using a hash function.
